(19) United States
(12) Patent Application Publication
(10) Pub. No.: US 2022/0183811 A1
(43) Pub. Date: Jun. 16, 2022

(54) ESTRUS DETERMINATION DEVICE FOR SOW, METHOD FOR DETERMINING ESTRUS OF SOW, AND PROGRAM FOR DETERMINING ESTRUS OF SOW

(71) Applicants: NH FOODS LTD., Osaka (JP); NTT DATA Corporation, Tokyo (JP)

(72) Inventors: Shin SUKEGAWA, Tsukuba (JP); Daisuke UCHIDA, Tsukuba-shi (JP); Masaki OKUDA, Tsukuba-shi (JP); Tatsuki YOSHIDA, Tokyo (JP); Naoki MORITA, Tokyo (JP); Yusuke OSHIRO, Tokyo (JP)

(21) Appl. No.: 17/653,506

(22) Filed: Mar. 4, 2022

Related U.S. Application Data
(63) Continuation of application No. PCT/JP2020/033004, filed on Sep. 1, 2020.

(30) Foreign Application Priority Data
Sep. 6, 2019 (JP) 2019-162720

(51) Int. Cl. A61D 17/00; A61B 10/00; A01K 29/00

(52) U.S. Cl. CPC A61D 17/002; A61B 2010/0029; A01K 29/005; A61B 10/0012

(57) ABSTRACT

Provided is a technology that allows estrus of a sow to be accurately determined without relying on experience or in intuition of an observer. An estrus determination device for a sow includes a measurement unit that measures, per unit time, a frequency of standing up and lying down of a sow raised in a stall and a determination unit that determines estrus of the sow on the basis of a plurality of frequencies repetitively measured by the measurement unit over a set given period.

### CROSS-REFERENCE TO RELATED APPLICATION

The present application is a continuation of International Application No. PCT/JP2020/0330004 filed on Sep. 1, 2020, the disclosures of which are hereby incorporated in their entirety by reference.

### TECHNICAL FIELD

The present invention relates to an estrus determination device for a sow, a method for determining estrus of a sow, and a program for determining estrus of a sow.

### BACKGROUND ART

A system, in which a sensor is installed in a facility for raising livestock to detect an abnormal behavior of the livestock, is known (see, e.g., Patent Document 1).

### CITATION LIST

### Patent Document

Patent Document 1: Patent Publication JP-A-2019-24482

### SUMMARY

### Technical Problem

In pig raising, determination of estrus of a sow is important from a viewpoint of increasing the number of piglets born per year, a viewpoint of maintaining reproductive cycles at appropriate intervals, and the like. However, even if a conventional technology allows an abnormal behavior of a target sow to be detected, the conventional technology cannot detect a specified state such as estrus. A large number of breeders have raised requests to accurately determine estrus of a sow without relying on experience or intuition.

The present invention has been made in order to solve such a problem and provides a technology of accurately determining estrus of a sow.

### Solution to Problem

An estrus determination device for a sow in a first aspect of the present invention includes: a measurement unit that measures, per unit time, a frequency of standing up and lying down of a sow raised in a stall; and a determination unit that determines estrus of the sow on the basis of a plurality of frequencies repetitively measured by the measurement unit over a set given period.

A method for determining estrus of a sow in a second aspect of the present invention includes: a measurement step of repetitively measuring, per unit time, a frequency of standing up and lying down of a sow raised in a stall over a set given period; and a determination step of determining estrus of the sow on the basis of a plurality of frequencies repetitively measured in the measurement step.

A program for determining estrus of a sow in a third aspect of the present invention causes a computer to execute: a measurement step of repetitively measuring, per unit time, a frequency of standing up and lying down of a sow raised in a stall over a set given period; and a determination step of determining estrus of the sow on the basis of a plurality of frequencies repetitively measured in the measurement step.

### Advantageous Effects of Invention

According to the present invention, it is possible to accurately determine estrus of a sow without relying on experience or intuition of an observer.

### BRIEF DESCRIPTION OF DRAWINGS

FIG. 1 is a diagram illustrating an overall view of a pig raising environment using a determination device according to the present embodiment.

FIG. 2 is a diagram illustrating a hardware configuration of the determination device.

FIGS. 3(A), 3(B), and 3(C) are diagrams illustrating a state of a sow in a stall.

FIG. 4 is a diagram illustrating a method of converting a frequency of standing up and lying down of the sow to a cumulative score.

FIG. 5 is a diagram illustrating transitions of the cumulative scores of sows not in estrus.

FIG. 6 is a diagram illustrating transitions of the cumulative scores of sows in estrus.

FIGS. 7(A), 7(B), and 7(C) are diagrams illustrating time blocks in which the frequency of standing up and lying down is to be measured.

FIG. 8 is a diagram illustrating different transitions of the cumulative scores due to different measurement time blocks.

FIG. 9 is a diagram illustrating a method of determining estrus using an estrus discriminator.

FIG. 10 is a diagram illustrating processing in a measurement step of measuring the cumulative score.

FIG. 11 is a diagram illustrating processing in a determination step of determining estrus of a target sow.

FIG. 12 is a diagram illustrating another estrus determination method.

### DESCRIPTION OF EMBODIMENTS

The following will describe the present invention through an embodiment of the present invention, but is not intended to limit the invention related to the scope of claims to the following embodiment. In addition, not all the configurations described in the embodiment are indispensable as means for solving the problem.

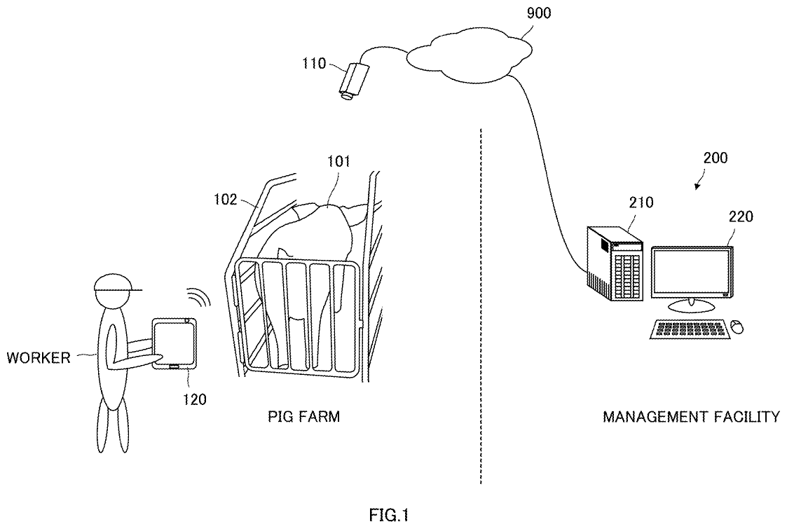

FIG. 1 is a diagram illustrating an overall view of a pig raising environment using a determination device 200 according to the present embodiment. In a pig farm, a sow 101 to be observed is housed in a stall 102. In the one stall 102, the one sow 101 is housed. The one stall 102 has such dimensions as to prevent the sexually mature sow 101 housed therein from circling by itself, which are, e.g., a width of about 80 cm and a depth of about 200 cm. Accordingly, motions of the sow 101 are limited to a standing-up motion and a lying-down motion. When there are a plurality of the sows 101 to be observed, a plurality of the stalls 102 in which the sows 101 are individually housed are provided in juxtaposition. Note that the dimensions of the stalls 102 can be set on the basis of the breed of the sows to be observed, individual differences between the sows, and a breeding environment.

A camera unit 110 includes an imaging sensor capable of overlooking and imaging the whole body of the sow 101 to be observed, converts an image resulting from the imaging by the imaging sensor to image data, and transmits the image data to a server 210 via an Internet 900. When there are the plurality of sows 101 to be observed, it may be possible to dispose the camera units 110 for the individual stalls 102 on a one-to-one basis or dispose the camera unit for each group of the plurality of stalls 102. When the camera unit is disposed for each group of the plurality of stalls 102, an angle of view of the camera unit 110 is adjusted so as to allow the sows 101 housed in the individual stalls 102 included in the group to be simultaneously observed.

In a management facility, a determination device 200 that determines estrus of the sow 101 to be observed is disposed. The determination device 200 includes the server 210, a display monitor 220 connected to the server 210, and the like, and the server 210 is connected to the Internet 900. The server 210 receives the image data transmitted from the camera unit 110 via the Internet 900, measures a frequency of standing up and lying down of the sow 101 from the image data, and determines whether or not the sow 101 is estrus on the basis of the measured frequency. The server 210 displays a result of the determination on the display monitor 220. When the determination result is requested by a worker working in the pig farm via a worker terminal 120, the determination result is displayed on a display unit of the worker terminal 120 via the Internet 900. Examples of the worker terminal 120 include a tablet terminal and a smartphone.

Note that a network connecting the camera unit 110 and the determination device 200 is not limited to the Internet 900, and may also be an intranet or the like. When the management facility is provided in the pig farm, near field communication may also be used.

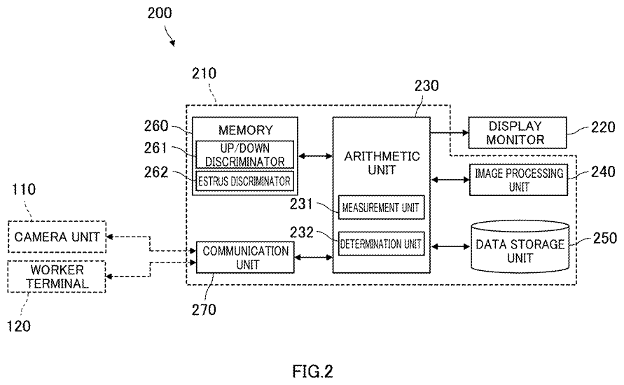

FIG. 2 is a diagram illustrating a hardware configuration of the determination device 200. As described above, the determination device 200 mainly includes the server 210 and the display monitor 220. The display monitor 220 includes, e.g., a liquid crystal panel, converts a video signal generated from an arithmetic unit 230 to a visually recognizable video, and displays the video. The server 210 mainly includes the arithmetic unit 230, an image processing unit 240, a data storage unit 250, a memory 260, and a communication unit 270.

The arithmetic unit 230 is, e.g., a CPU and executes various programs read from the memory 260 to control the entire determination device 200 or execute various arithmetic processing. For example, when executing processing as a measurement unit 231, the arithmetic unit 230 cooperates with the image processing unit 240 to measure the frequency of standing up and lying down of the target sow 101 per unit time. When executing processing as a determination unit 232, the arithmetic unit 230 uses a result of the measurement by the measurement unit 231 to determine estrus of the sow 101 and outputs a result of the determination to the display monitor 220 or the worker terminal 120. Specific processing will be described later in detail.

The image processing unit 240 is, e.g., an ASIC for image processing and executes image processing such as generating a posture determination image by cutting out an image region of the target sow from the image data received from the camera unit 110. The data storage unit 250 is, e.g., an HDD (Hard Disc Drive) and stores identification information of the sow 101 to be observed and a cumulative score associated with the identification information. The cumulative score is obtained by quantifying up/down states of the target sow 101 observed during a predetermined unit time and cumulatively adding the resulting quantities. The cumulative score is counted every day while the target sow 101 is housed in the stall 102, and the cumulative score is recorded together with date information in the data storage unit 250.

The memory 260 is, e.g., an SSD (Solid State Drive) and stores not only a control program for controlling the determination device 200 and an estrus determination program for determining estrus of the target sow 101, but also various parameter values, functions, a lookup table, and the like. In particular, an up/down discriminator 261 and an estrus discriminator 262, each of which is a learned model, are stored. The up/down discriminator 261 discriminates the up/down state of the sow seen in the posture determination image input thereto. The estrus discriminator 262 discriminates, from the daily cumulative score input thereto, whether or not the sow is in estrus, details of which will be described later.

The communication unit 270 is, e.g., a wired LAN unit. The arithmetic unit 230 requests the image data from the camera unit 110 connected to the Internet 900 via the communication unit 270, and receives the image data transmitted from the camera unit 110 in response to the request. The determination unit 232 also transmits, in response to a request from the worker terminal 120 received via the communication unit 270, a result of determining the estrus to the communication unit 270.

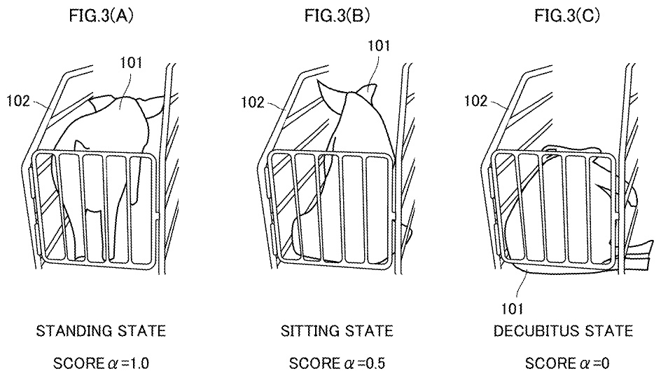

FIG. 3 is a diagram illustrating a state of the sow 101 in the stall 102. In the present embodiment, the up/down state of the sow 101 to be observed is identified as any of a decubitus state, a sitting state, and a standing state.

FIG. 3(A) illustrates the sow 101 in the standing state. The standing state is a state of the sow 101 in a posture in which the sow 101 is standing on both front and rear legs. In the present embodiment, when the standing state is observed, a score α=1.0 is given. FIG. 3(B) illustrates the sow 101 in the sitting state. The sitting state is a state of the sow 101 in a posture in which the sow 101 is on either one of the front legs and the rear legs, while the other legs are bent. The figure illustrates the sow 101 having the rear legs bent, while having buttocks in contact with the ground. In the present embodiment, when the sitting state is observed, a score α=0.5 is given. FIG. 3(C) illustrates the sow 101 in the decubitus state. The decubitus state is a state where the sow 101 in a posture in which both the front legs and the rear legs are bent or extended sideways, and no load is placed on the legs. The figure illustrates the sow 101 having all the legs extended sideways, while having a flank in contact with the ground. In the present embodiment, when the decubitus state is observed, the score α=0 is assumedly satisfied.

The camera unit 110 periodically images the sow 101 in such a varying state under the control of the measurement unit 231. The measurement unit 231 acquires the image data transmitted from the camera unit 110 and gives the image data to the image processing unit 240. The image processing unit 240 cuts, out of the image data, a region of the sow 101 to be observed and performs preset image processing thereon to generate the posture determination image. The preset image processing is adjustment of an image size, contour enhancement around a specific color (e.g., a color of skin of the target sow), or the like. The measurement unit 231 reads the up/down discriminator 261 from the memory 260 and inputs the posture determination image generated by the image processing unit 240 thereto. The up/down discriminator 261 outputs any of the standing state, the sitting state, and the decubitus state as a result of identifying the up/down state of the sow seen in the posture determination image. The measurement unit 231 causes the score α to be fixed depending on the result of the identification.

The up/down discriminator 261 is the learned model learned by machine learning. The up/down discriminator 261 is produced in advance by a learning device. Specifically, to the learning device, a large number of teacher data items each as a set of the posture determination image and a correct answer thereof (any of the standing state, the sitting state, and the decubitus state) are given, and the learning device executes supervised learning as a type of machine learning. For the supervised learning, a CNN (convolutional neural network) appropriate for image recognition is used herein. The up/down discriminator 261 that has finished learning by the supervised learning is moved from the learning device to the memory 260 to be subjected to the use described above.

As a unit time T₀, e.g., two hours is set, and how many times the target sow 101 changed the up/down states during the two hours is evaluated. In other words, the frequency of standing up and lying down is evaluated. As an evaluation value for the evaluation, when the target sow 101 gets up once, the score α=1.0 is given as described above and, when the target sow 101 sits down once, the score α=0.5 is given. The number of times the target sow 101 lies down is not counted as the evaluation value, since the score α=0 is satisfied. The cumulative score thus accumulated during the unit time T₀ can serve as the evaluation value representing the frequency of standing up and lying down of the target sow 101 on a measurement day.

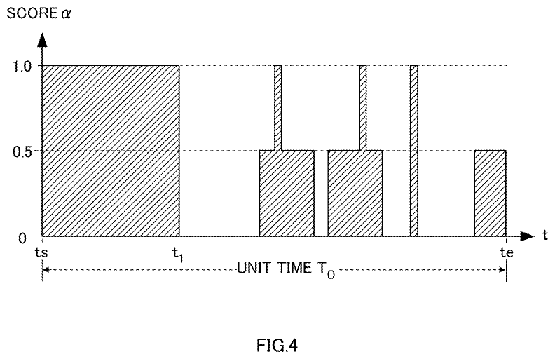

In the present embodiment, to further enhance the accuracy of the evaluation value, the evaluation value is calculated in consideration also of a time during which each of the standing state, the sitting state, and the decubitus state continued. In other words, a method that corrects the accumulated score on the basis of the time during which each of the decubitus state, the sitting state, and the standing state continued is used. FIG. 4 is a diagram illustrating a method of converting the frequency of standing up and lying down of the target sow 101 to the cumulative score in the present embodiment. The abscissa axis represents an elapsed time, while the ordinate axis represents the score α corresponding to standing up (α=1.0), sitting down (α=0.5), and lying down (α=0).

The figure illustrates an example of an observation result obtained by starting to observe the target sow 101 at a time ts and continuing the observation till a time te at which the unit time T₀ elapses. The camera unit 110 images the sow 101 at, e.g., 1 frame/second, and transmits the resulting image data items to the determination device 200. The measurement unit 231 uses the individual image data items received thereby to identify in which one of the standing, sitting, and decubitus states the sow 101 is in and causes the score α to be fixed. In other words, the measurement unit 231 causes the score α corresponding to the state of the sow 101 to be fixed every second. Then, the measurement unit 231 adds the current score α to the cumulative score since the time ts to update the cumulative score. The measurement unit 231 continues this processing till the time te at which the unit time T₀ elapses.

By thus counting the cumulative score, the measurement unit 231 can calculate the evaluation value representing the frequency of standing up and lying down considering the time during which each of the standing state, the sitting state, and the decubitus state was continued. For example, in the figure, the standing state (α=1.0) is continued from the time ts to a time t₁ but, when a period from the time ts to the time t₁ is assumed to be 1000 seconds, 1.0 is added every second, and accordingly 1.0×1000=1000 is added to the cumulative score.

It can be said that the cumulative score thus calculated represents an intensity of activity of the sow 101. In other words, when the cumulative score is large, it can be said that the sow 101 actively moved and, when the cumulative score is small, it can be said that the sow 101 stayed inactive. When the measurement unit 231 calculates the cumulative score of the target sow 101 in a determined time block every day over a given period and stores the cumulative score in the data storage unit 250 every day, it is possible to find changes in the intensity of activity of the sow 101 during the period.

In terms of increasing the number of piglets born of the sow per year and maintaining reproductive cycles at appropriate intervals, it is important to accurately determine estrus of the sow housed in the stall. Thus far, the determination of whether or not a sow is in estrus has largely relied on experience or intuition of a skilled worker. Consequently, a large number of breeders have raised requests to accurately determine estrus of the sow without relying on the experience or intuition of the skilled worker. Under such a background, the present inventors have continued study and found that there is a correlation between a change in the intensity of activity of the sow and estrus. At the same time, the present inventors have developed a method of evaluating the intensity of activity as described above. The relationship between the change in the intensity of activity and the estrus will be described using the cumulative score described above.

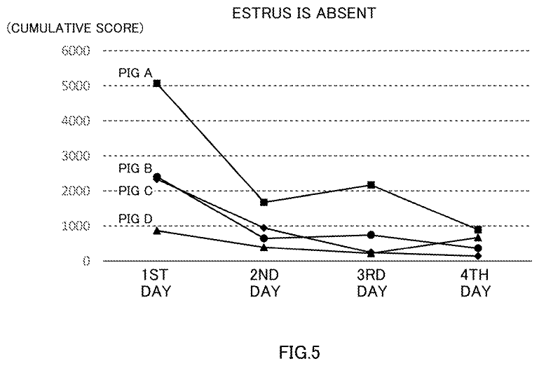

FIG. 5 is a diagram illustrating transitions of the cumulative scores of sows not in estrus. The abscissa axis represents the number of elapsed observation days, while the ordinate axis represents the cumulative scores on each of the observation days. Results of observing four sows (sow A, sow B, sow C, and sow D) were plotted herein.

Each of the sows has its own personality. One of the sows inherently moves actively, while another of the sows is inactive. Accordingly, the observation day on which each of the sows exhibited the cumulative score on the basis of which it can be considered that the sow moved more actively than on the other observation days is assumed to be the first day, and the cumulative score was plotted. Then, the cumulative scores on the three days subsequent thereto were plotted, and a change in the cumulative score over a total of four days was shown.

As illustrated in the figure, the transitions of the cumulative scores of the four sows are rightwardly decreasing or remain flat. No estrus was observed in any of the four sows. In the example shown herein, the number of the sows was four, but transitions of the cumulative scores of other sows in which no estrus was observed had approximately the same results.

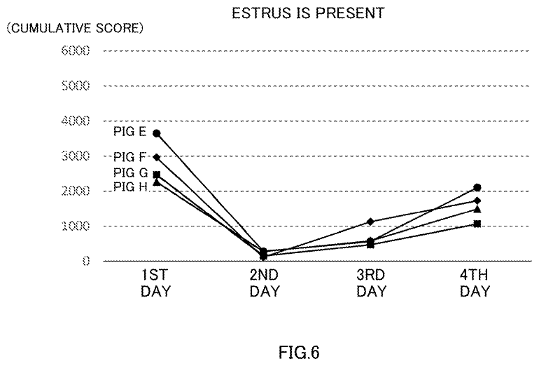

FIG. 6 is a diagram illustrating transitions of the cumulative scores of sows in estrus. In the same manner as in FIG. 5, the abscissa axis represents the number of elapsed observation days, while the ordinate axis represents the cumulative score on each of the observation days. Results of observing four sows (sow E, sow F, sow G, and sow H) were also plotted herein.

In the case of FIG. 6 also, in the same manner as in the case of FIG. 5, the observation day on which each of the sows exhibited the cumulative score on the basis of which it can be considered that the sow moved more actively than on the other observation days is assumed to be the first day, and the cumulative score was plotted. Then, the cumulative scores on the three days subsequent thereto were plotted, and a change in the cumulative score over a total of four days was shown.

As illustrated in the figure, each of the transitions of the cumulative scores of these four sows exhibits a V-shaped shape in which the cumulative score temporarily decreased, and then recovered. Estrus was observed in each of the four sows. In the example shown herein, the number of the sows was four, but transitions of the cumulative scores of other sows in which the estrus was observed had approximately the same results. Note that the present inventors used transition data of 181 cumulative scores obtained from 105 sows to perform verification in FIGS. 5 and 6.

From a result of the foregoing verification, the present inventors obtained findings such that, when a change in the intensity of activity over a given period exhibits a V-shaped shape, it is highly possible that the sow is in estrus. In other words, by measuring and storing the cumulative score of the sow to be observed every day as described above, extracting the cumulative scores during the given period at the time at which it is desired to know the presence or absence of estrus, and checking a change in the cumulative score, it is possible to determine whether or not the sow is in estrus.

As the given period over which the measurement unit 231 repetitively measures the cumulative score, three or more days which allow a V-shaped shape to be recognized first are required and, in consideration of a case where the cumulative score has a bottom value on the second day, the third day, or the fourth day, the given period is preferably set to a maximum of about seven days or less. In addition, determining a length of the unit time T₀ described above, in which the frequency of standing up and lying down of the sow is to be measured, and the time of the day, at which the unit time T₀ is to be set, is also important in increasing the accuracy of the determination. First, a description will be given of the determination of the time of the day at which the unit time T₀ is to be set. Note that, in the following description, the unit time T₀ may be referred to also as a measurement time T₀ as a time during which the frequency of standing up and lying down is to be measured.

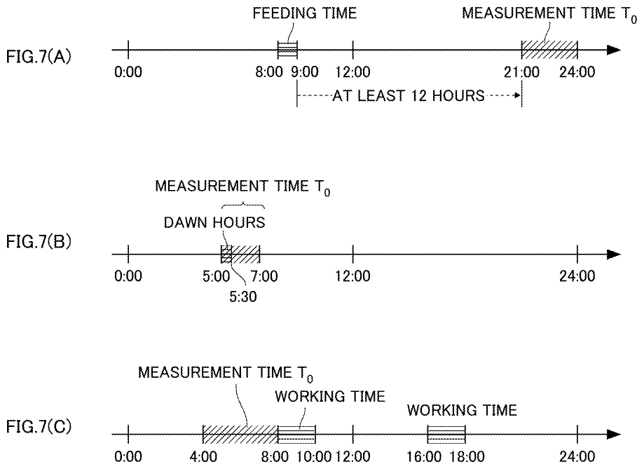

FIG. 7 is a diagram illustrating time blocks in which the frequency of standing up and lying down is to be measured. In principle, movement of a sow to be observed such as spontaneous standing up and lying down under no external influence is to be measured, and therefore it can be said that the measurement time T₀ is preferably set in a quiet time block in accordance with the rhythm of a natural environment. FIG. 7(A) is a diagram illustrating a preferred relationship between a feeding time during which the sow is to be fed and the measurement time T₀. The feeding time is a time block in which the sow actively moves irrespective of the presence or absence of estrus. In addition, even after the feeding, the sow actively moves for a while. Accordingly, the measurement time T₀ is preferably set within a period after a lapse of twelve hours from the feeding of the sow and before next feeding of the sow. In the example in the figure, a period between 8:00 and 9:00 is set as the feeding time, while three hours between 21:00 twelve hours after 9:00 and 24:00 is set as the measurement time T₀.

FIG. 7(B) is a diagram illustrating a preferred relationship between dawn hours in the vicinity of the pig farm and the measurement time T₀. It is assumed herein that a window is provided in the pig farm and, in the farm, a dark state shifts to a bright state at dawn. In general, the movement of a sow stagnates in the dark state, and the sow gradually begins to move when it gets bright. Accordingly, the measurement time T₀ is preferably set to include the dawn hours during which an environment in which the stall is placed shifts from the dark state to the bright state. In the example in the figure, when the dawn hours are from 5:00 to 5:30, two hours from 5:00 to 7:00 including the dawn hours is set as the measurement time T₀. Note that, when the pig farm has no window and indoor brightness is controlled by lighting, the measurement time T₀ may appropriately be set so as to include a time block in which the environment in which the stall is placed is brought by lighting from the dark state into the bright state.

FIG. 7(C) is a diagram illustrating a preferred relationship between a working time during which a worker works in the pig farm and the measurement time T₀. The working time is a time during which the worker cleans up the pig farm or moves around the farm, while checking health conditions of the raised sows, and corresponds to a time block in which the sows actively move irrespective of the presence or absence of estrus. Accordingly, the measurement time T₀ is preferably set within a period during which there is no person in an environment around where the stall is placed. In the example in the figure, the working times are set between 8:00 and 10:00 and between 16:00 and 18:00, while four hours between 4:00 and 8:00 is set as the measurement time T₀.

As the measurement time T₀, three hours, two hours, and four hours are respectively set in the example of FIG. 7(A), the example of FIG. 7(B), and the example of FIG. 7(C). However, a specific length of time to be set as the measurement time T₀ may be determined appropriately in relation to a factor to be considered such as the feeding time. It has been found through experiment that, in either case, the measurement time T₀ is preferably a time of two hours or longer and six hours or less.

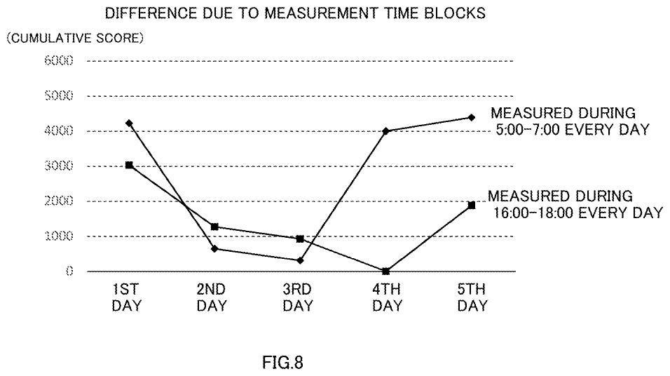

Next, a description will be given of how different measurement time blocks show up in the transitions of the cumulative scores. FIG. 8 is a diagram illustrating the different transitions of the cumulative scores due to the different measurement time blocks. The abscissa axis represents the number of elapsed observation days, while the ordinate axis represents the cumulative score on each of the observation days. What is shown herein is a transition that is obtained by plotting the cumulative scores measured every day during a period from 5:00 to 7:00 for a specified sow exhibiting estrus and connecting the plots and a transition that is obtained by plotting the cumulative scores measured every day during a period from 16:00 to 18:00 for a specified sow exhibiting estrus and connecting the plots.

The period from 5:00 to 7:00 is a preferred time block in each of FIGS. 7(A) to 7(C), while the period from 16:00 to 18:00 is a time block in which influence of feeding remains and which overlaps the working time of the worker. The transition of the cumulative score measured during the period from 16:00 to 18:00 also exhibits a V-shaped shape for sure, but a change in the transition is gentler than that in the transition of the cumulative score measured during the period from 5:00 to 7:00. In a determination method described later, as the change in the V-shaped shape appears to be more marked, the estrus can more accurately be determined and, accordingly, it can be said that the measurement time T₀ is more preferably set to the time block from 5:00 to 7:00 than to that from 16:00 to 18:00.

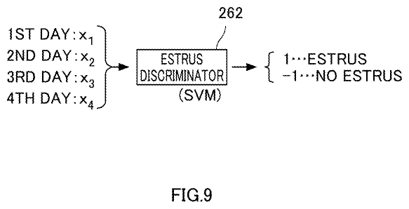

When the measurement unit 231 observes the up/down state of the target sow 101 in the determined time block every day, calculates the cumulative score, and stores the cumulative score in the data storage unit 250, the determination unit 232 can determine, in response to a request from a user, the presence or absence of estrus of the target sow 101. In the present embodiment, the determination unit 232 uses the estrus discriminator 262 to determine the presence or absence of the estrus. FIG. 9 is a diagram illustrating a method of determining estrus using the estrus discriminator 262.

The determination unit 232 reads the estrus discriminator 262 from the memory 260 and inputs thereto the cumulative scores during a given period that have been stored in the data storage unit 250. It is assumed herein that the given period is four days, and a four-dimensional vector (x₁, x₂, x₃, x₄) including a cumulative score x₁ on the first day, a cumulative score x₂ on the second day, a cumulative score x₃ on the third day, and a cumulative score x₄ on the fourth day is input to the estrus discriminator 262.

The estrus discriminator 262 in the present embodiment is a support vector machine (SVM), and is a learned model learned in advance by a learning device. Specifically, the cumulative scores during the given period are given as an input vector together with a correct answer label “ESTRUS IS PRESENT” or “ESTRUS IS ABSENT”, and a discriminant function that separates a space in which “ESTRUS IS PRESENT” and a space in which “ESTRUS IS ABSENT” from each other is fixed by learning. The estrus discriminator 262 produced through such learning is moved from the learning device to the memory 260 to be used for the determination.

When the determination unit 232 inputs the four-dimensional vector (x₁, x₂, x₃, x₄) to the estrus discriminator 262, the estrus discriminator 262 outputs “1” representing that “ESTRUS IS PRESENT” or “−1” representing that “ESTRUS IS ABSENT”. The determination unit 232 outputs a result of the determination, which is the output from the estrus discriminator 262, to the display monitor 220 or the worker terminal 120. For example, on the display monitor 220, a management number of the target sow 101 and the presence or absence of estrus, such as “MANAGEMENT NO. x x/ESTRUS IS PRESENT”, are displayed together. Note that the cumulative scores during the four days are input as the four-dimensional vector herein, but it is also possible to produce a SVM by adding another feature value to the input vector in addition to the cumulative score. For example, it is possible to produce a SVM to which an ablactation day or an ambient temperature is input as the feature value. In addition, the learned model is not limited to the SVM that outputs a dichotomous determination which is either “ESTRUS IS PRESENT” or “ESTRUS IS ABSENT”, and may also output a multi-stage determination such as, e.g., “80% ESTRUS PROBABILITY”.

As described above, the determination device 200 in the present embodiment executes two major processing steps, i.e., a measurement step of measuring the cumulative score and a determination step of determining estrus of the target sow. So, respective flows of processing will be summarized.

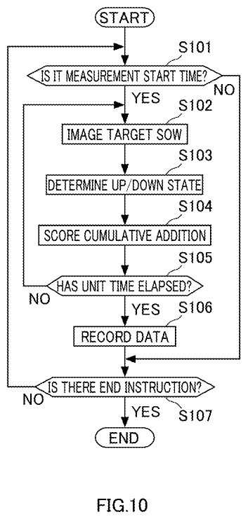

FIG. 10 is a flow chart illustrating the processing in the measurement step of measuring the cumulative score. The flow is started at the time when the sow 101 to be observed is housed in the stall 102 and an instruction to start continuous observation is given by a system operator.

The measurement unit 231 checks a current time in Step S101, and determines whether or not a preset measurement start time is reached. When determining that the measurement start time is reached, the measurement unit 231 advances to Step S102 and, when determining that the measurement start time is not reached, the measurement unit 231 advances to Step S107. When having advanced to Step S102, the measurement unit 231 transmits an instruction signal representing an instruction for imaging to the camera unit 110. When receiving the instruction signal, the camera unit 110 executes imaging of the sow 101 and transmits generated image data to the measurement unit 231.

When receiving the image data from the camera unit 110, the measurement unit 231 advances to Step S103, gives the image data to the image processing unit 240 to cause the image processing unit 240 to generate the posture determination image, and inputs the generated posture determination image to the up/down discriminator 261. Then, when the up/down discriminator 261 outputs any of the standing state, the sitting state, and the decubitus state as a result of identification, the measurement unit 231 causes the score α to be fixed depending on the result of the identification.

In Step S104, the measurement unit 231 adds the score α fixed this time to the previous cumulative score to update the cumulative score. Then, the measurement unit 231 advances to Step S105, checks the current time, and determines whether or not the preset unit time T₀ has elapsed from the measurement start time. When determining that the unit time T₀ has elapsed, the measurement unit 231 advances to Step S106 and, when determining that the unit time T₀ has not elapsed, the measurement unit 231 returns to Step S102 to continue the observation of the sow 101. Note that, when returning to Step S102 and transmitting the instruction signal representing the instruction for imaging to the camera unit 110 again, the measurement unit 231 adjusts timing to provide a preset imaging period (e.g., 1 second).

When having advanced to Step S106, the measurement unit 231 causes the cumulative score to be fixed and records the cumulative score together with identification information of the sow 101 and date information in the data storage unit 250. In Step S107, the arithmetic unit 230 checks whether or not an instruction to end the sequential measurement step processing is issued. The end instruction is issued through a menu operation by the system operator or through an end determination by the control program. When the end instruction is not issued, the measurement unit 231 returns to Step S101 and continues the sequential processing and, when the end instruction is not issued, the measurement unit 231 ends the processing in the measurement step.

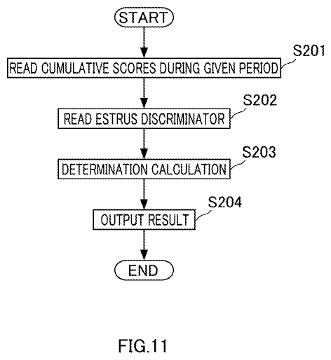

FIG. 11 is a diagram illustrating the processing in the determination step of determining estrus of the target sow. The determination step is started in response to a request from the system operator or the worker. The system operator or the worker operates the determination device 200 or the worker terminal 120 to specify the target sow for which it is desired to know whether the estrus is present or absent. The flow is started at the time when the sow serving as a determination target is specified.

In Step S201, the determination unit 232 reads, from the data storage unit 250, the cumulative scores of the specified sow during o a most recent given period. Specifically, when the given period is set to, e.g., four days, the cumulative scores during the most recent four days are read. The determination unit 232 advances to Step S202, and reads the estrus discriminator 262 from the memory 260. Then, in Step S203, the determination unit 232 inputs the read cumulative scores during the given period that have been read from the data storage unit 250 to the estrus discriminator 262 read from the memory 260, and executes determination calculation. When an output representing “ESTRUS IS PRESENT” or “ESTRUS IS ABSENT” is obtained as a result of the determination calculation, in Step S204, the determination unit 232 outputs a result of the determination to the display monitor 220 or the worker terminal 120, and ends the sequential processing.

Note that, in the example described herein, the processing in the determination step is executed in response to the request from the system operator or the worker but, when a predetermined condition is satisfied, the processing in the determination step may also be automatically executed. For example, the determination unit 232 may also execute the processing in the determination step on each of the sows to be observed at a predetermined time every day, produce a list of a group of sows for which it is determined that “ESTRUS IS PRESENT” and a group of sows for which it is determined that “ESTRUS IS ABSENT”, and output the list to the display monitor 220 or the worker terminal 120. Alternatively, the determination unit 232 may also automatically execute the processing in the determination step with timing with which the daily cumulative score is recorded in the data storage unit 250, and notify the system operator or the worker only when it is determined that “ESTRUS IS PRESENT”.

In the present embodiment described heretofore, the estrus is determined using the estrus discriminator 262 which is the SVM, but the estrus discriminator 262 is not limited to the SVM. As a discriminator to be generated by machine learning, another method such as logistic regression or random forest can also be used. Alternatively, it may also be possible to use a method that analytically determines estrus instead of using the discriminator.

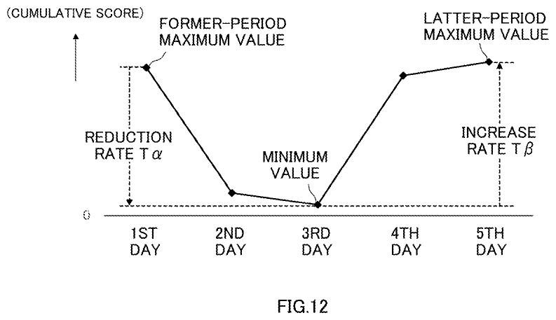

FIG. 12 is a diagram illustrating an estrus determination method that analytically determines estrus. The abscissa axis represents the number of elapsed observation days, while the ordinate axis represents the cumulative score on each of the observation days. What is shown herein is a transition obtained by plotting the cumulative scores of the specified sow exhibiting estrus on the first to fifth days and connecting the plots. As described above, the sow in estrus has the V-shaped change in the intensity of activity over a given period, and therefore it is appropriate to analyze whether or not the transition of the cumulative score has a V-shaped shape to determine whether or not the sow is in estrus.

Accordingly, it is checked whether or not the cumulative score has a minimum value on any of middle days (which are the second to fourth days in the case of the figure) in the given period (which is five days in the case of the figure). When the minimum value is not present on any of the middle days, it is determined that “ESTRUS IS ABSENT”. When the minimum value is present on any of the middle days, it is assumed that a period before the observation day on which the minimum value was measured is a former period and a period thereafter is a latter period. In the case of the figure, the cumulative score on the third day has a minimum value, and therefore the first and second days are in the former period, while the fourth and fifth days are in the latter period.

Then, a former-period maximum value which is a maximum value of the cumulative score during the former period and a latter-period maximum value which is a maximum value of the cumulative score during the latter period are determined. In the case of the figure, the cumulative score on the first day has the former-period maximum value, while the cumulative score on the fifth day has the latter-period maximum value. It is analyzed whether or not a change from the former-period maximum value to the latter-period maximum value through a minimum value has a V-shaped shape sufficient to allow a determination that “ESTRUS IS PRESENT” to be made. Specifically, when a reduction rate Tα from the former-period maximum value to the minimum value is larger than a preset reference reduction rate Tα₀ and an increase rate Tβ from the minimum value to the latter-period maximum value is larger than a preset reference increase rate Tβ₀, it is determined that “ESTRUS IS PRESENT”. Otherwise, it is determined that “ESTRUS IS ABSENT”. Note that the reference reduction rate Tα₀ and the reference increase rate Tβ₀ are set using fixed data having a known result.

While the several determination methods have been described heretofore, which one of the determination methods is to be used in the determination device can be determined on the basis of the number and breed of the sows to be observed, a scale of the determination device, cost, required determination accuracy, and the like. In addition, not only the determination method, but also a method of measuring the up/down state is not limited to the method described above. For example, it may also be possible that a distance sensor that measures heights in the vicinity of a head and buttocks of the sow 101 housed in the stall 102 is placed on a ceiling, instead of the camera unit 110, and the measurement unit 231 identifies the up/down state based on an output from the distance sensor.

Also, in the present embodiment, not only the standing state and the decubitus state, but also the sitting state is regarded as a target of measurement of the frequency of standing up and lying down, and the score α=0.5 is given to the sitting state. However, it may also be possible to adjust the numerical value of the score to a value other than 0.5 in consideration of the breed, body weight, or the like of the sow or further divide the sitting state depending on the posture into subcategories and give scores of different values to the individual subcategories. Conversely, when the sow is of a breed having short legs or when the determination accuracy need not be so high, the frequency of standing up and lying down may also be measured by omitting the sitting state and using only the two states, i.e., the standing state and the decubitus state.

### REFERENCE SIGNS LIST

101 Sow
102 Stall
110 Camera unit
120 Worker terminal
200 Determination device
210 Server
220 Display monitor
230 Arithmetic Unit
231 Measurement unit
232 Determination unit
240 Image processing unit
250 Data storage unit
260 Memory
261 Up/down discriminator
262 Estrus discriminator
270 Communication unit
900 Internet

What is claimed is:

1. An estrus determination device for a sow, the device comprising:
a measurement unit that measures, per unit time, a frequency of standing up and lying down of a sow raised in a stall; and
a determination unit that determines estrus of the sow on the basis of a plurality of frequencies repetitively measured by the measurement unit over a preset given period.

2. The estrus determination device for a sow according to claim 1, wherein the measurement unit identifies, as an up/down state of the sow, any of a decubitus state, a sitting state, and a standing state and uses, as the frequency, a cumulative value obtained by cumulatively adding scores respectively set in advance to the individual identified states.

3. The estrus determination device for a sow according to claim 2, wherein the measurement unit corrects the score on the basis of a time during which each of the decubitus state, the sitting state, and the standing state was continued.

4. The estrus determination device for a sow according to claim 1, wherein the given period is a period of three days or longer and seven days or less, while the unit time is a time of two hours or longer and six hours or less which is set every day during the given period.

5. The estrus determination device for a sow according to claim 1, wherein the unit time is set within a period after a lapse of twelve hours from feeding of the sow and before next feeding of the sow.

6. The estrus determination device for a sow according to claim 1, wherein the unit time is set so as to include a time block in which an environment in which the stall is placed shifts from a dark state to a bright state.

7. The estrus determination device for a sow according to claim 1, wherein the unit time is set within a period during which no person is present in an environment around where the stall is placed.

8. The estrus determination device for a sow according to claim 1, wherein the determination unit determines estrus of the sow by using a discriminator preliminarily subjected to machine learning to discriminate estrus of the sow by using the plurality of frequencies repetitively measured over the given period.

9. The estrus determination device for a sow according to claim 8, wherein the discriminator is a support vector machine.

10. The estrus determination device for a sow according to claim 1, wherein the determination unit determines that the sow is in estrus when the frequency in the given period exhibits a change of a temporarily decrease and then an increase again.

11. A method for determining estrus of a sow, the method comprising:
a measurement step of repetitively measuring, per unit time, a frequency of standing up and lying down of a sow raised in a stall over a set given period; and
a determination step of determining estrus of the sow on the basis of a plurality of frequencies repetitively measured in the measurement step.

12. A program for determining estrus of a sow, the program causing a computer to execute:
a measurement step of repetitively measuring, per unit time, a frequency of standing up and lying down of a sow raised in a stall over a set given period; and
a determination step of determining estrus of the sow on the basis of a plurality of frequencies repetitively measured in the measurement step.
